(19) United States
(12) Patent Application Publication
(10) Pub. No.: US 2024/0242790 A1
(43) Pub. Date: Jul. 18, 2024

(54) SYSTEM FOR ASSISTING WITH PROVISION OF DIAGNOSTIC INFORMATION

(71) Applicant: Valeo Systemes Thermiques, Le Mesnil-Saint-Denis (FR)

(72) Inventor: Georges De Pelsemaeker, Le Mesnil-Saint-Denis (FR)

(73) Assignee: Valeo Systemes Thermiques, Le Mesnil-Saint-Denis (FR)

(21) Appl. No.: 18/559,390

(22) PCT Filed: Apr. 26, 2022
(86) PCT No.: PCT/EP2022/060976

§ 371 Date: Nov. 7, 2023

(30) Foreign Application Priority Data
May 7, 2021 (FR) FR2104824

(51) Int. Cl. G16H 10/20; A61B 5/00

(52) U.S. Cl. CPC G16H 10/20; A61B 5/6893; A61B 5/7221; A61B 5/7275; A61B 5/742

(57) ABSTRACT

The present invention relates to a system (1) for assisting with provision of diagnostic information, especially with a view to detecting a disease in a person, especially a contagious disease such as COVID-19, this detecting system comprising: —a device (7) for acquiring examination data on the person, this acquiring device especially comprising, to acquire these examination data, at least a sensor, such as a radar, for measuring physiology and a thermal camera, this acquiring device comprising a plurality of sensors arranged to operate without contact with the person, at least one of the contactless sensors being arranged to acquire a response datum provided by the person.

The present invention relates to a system for assisting with the provision of diagnostic information, especially with a view to detecting a disease in a person, especially a contagious disease such as COVID-19.

Today, the detection of potential COVID-19 patients is carried out by taking the temperature, conducting complementary clinical examinations, then by administering a COVID-19 test, which still has a relatively low confidence level. A lung scan can confirm the severity of the disease with a good precision level.

There is a significant need to be able to detect the potential patients of a contagious disease, for example the disease linked to COVID-19, rapidly, in particular using mobile means.

The invention thus relates to a system for detecting a disease in a person, especially a contagious disease such as COVID-19, this detecting system comprising:
a device for acquiring examination data on the person, this acquiring device especially comprising, to acquire these examination data, at least a sensor, such as a radar, for measuring physiology and a thermal camera,
a data processing device arranged to receive these examination data obtained by the acquiring device,
a display device arranged to display diagnostic information for the disease based on an analysis of said examination data, this diagnostic information being able to be representative of a probability level that the person has the disease.

According to one aspect of the invention, the data processing device comprises an artificial intelligence unit arranged to process the examination data obtained by the acquiring device and to provide said diagnostic information.

According to one aspect of the invention, the data processing device and the display device are part of the same apparatus, for example a computer, in particular a laptop computer.

In this case, the display device is a screen of the laptop computer and the data processing device comprises microprocessor of this computer.

In a variant, the display device is arranged to be visible by the examined person, and in particular the display device, which in particular comprises a screen, is remote from the data processing device, the display device and the data processing device being for example connected to one another by a wireless link, for example by a 3G, 4G or 5G communication protocol, or by the Internet, or by WiFi, for example.

According to one aspect of the invention, the display device, and optionally also the data processing device, are arranged to be embedded on a motor vehicle.

In a variant, the display device, and optionally also the data processing device, are arranged to be placed in a fixed manner, in a building or an outer courtyard, for example.

According to one aspect of the invention, the processing device is arranged to perform the diagnosis automatically, without human intervention.

Due to automatic processing, the invention allows rapid diagnosis and/or mass screening, allowing a faster return to work or lockdown lift.

According to one aspect of the invention, the device for acquiring examination data is arranged to allow contactless measurements at a safe distance of vital signs as well as thermal and visible imaging on the person to be examined.

The system according to the invention thus advantageously merges contactless measurements at a safe distance of vital signs and thermal and visible imaging.

According to one aspect of the invention, the artificial intelligence unit is arranged to use a diagnostic model based on artificial intelligence and fed by a reasonable number of clinical measurements.

Due to relatively lightweight equipment, the invention in particular allows easy deployment of field hospitals to support populations.

The invention allows the performance of mobile diagnoses. It is easy to implement, for example using a thermal camera, a physiological measurement sensor such as a radar, and a personal laptop computer. The invention enables rapid implantation over a whole territory.

According to one aspect of the invention, the acquiring device comprises a radar for acquiring data relating to the vital signs of the person, a thermal camera for temperature measurements providing temperature data, and a camera operating in the visible spectrum for characterizing the tested person providing data characterizing the person.

According to one aspect of the invention, the data processing device is arranged to run a diagnostic algorithm based on a fusion of vital data, especially a respiration rate, a respiration depth, an inhalation and exhalation time, a heart rhythm and an arrhythmia.

According to one aspect of the invention, the algorithm uses temperatures measured in remarkable areas, located by processing of the image, or the oxygen level linked to the examined person.

Preferably, these remarkable temperature measurement areas are located inside the mouth, on the tip of the nose, on the cheekbones and on the palm of the hand.

According to one aspect of the invention, the diagnostic algorithm uses a characterization of the person, such as age, gender, clothing, height, body mass index, also called BMI.

According to one aspect of the invention, the system is arranged to acquire examination data until the diagnostic information is available within a period of time in particular between 30 and 120 seconds.

According to one aspect of the invention, the system is arranged to allow acquisition of the examination data by taking measurements at a distance of 60 cm to 2 m between the acquiring device and the person. The person is prevented from having to be in contact with the acquiring device.

The invention thus allows rapid diagnosis without additional delivery time to a physician, for example. The diagnostic information may, if necessary, be sent automatically to a physician and can be stored on a Cloud-type data storage system.

According to one aspect of the invention, the data processing device uses an algorithm for analyzing the acquired examination data and, if applicable, sorting people in order to detect the cases of patients who are sick, based on all of the collected data and artificial intelligence whose first level of learning is carried out on a sample in a hospital environment.

This learning by the artificial intelligence can be carried out via a set of measurements collected by the system, but also via the medical monitoring of patients. This allows the model to be improved over time.

According to one aspect of the invention, the measurements carried out by the acquiring device can be used to subsequently refine the diagnosis made by the artificial intelligence.

Owing to the invention, a diagnosis can made without contact with the person, which limits the risks of contamination, which is particularly advantageous for example in the case of a pandemic such as that linked to COVID-19.

According to one aspect of the invention, the measured examination data comprise at least one of the following data: temperatures measured at different points of the body of the person to be examined, a respiratory characteristic or a cardiac characteristic.

According to one aspect of the invention, the acquiring device is arranged to acquire examination data comprising an outside temperature, a temperature measured on a cheekbone of the person, a temperature measured on the tip of the person's nose, and also if applicable, a maximum temperature of the face and a temperature of a clothing item or a reference temperature-controlled surface.

According to one aspect of the invention, the remarkable measurement points are located by an artificial intelligence via an object identification flowchart.

According to one aspect of the invention, the temperature relative to a remarkable point is obtained by time average and by average of the temperatures of a surface defined by pixels from an infrared camera image near the identified remarkable point on the image visible via an object identification algorithm.

According to one aspect of the invention, the identification of the personal characterizations is done by means of Red Green Blue camera (or RGB camera) or Far InfraRed camera (FIR camera) in addition to a reading of the person's identity, owing to a classification system whose training can be carried out on RGB (Red Green Blue) or Infrared images. A larger number of parameters, in particular the age, the gender, the height, the body mass index, the phenotype, for example, are used to improve the diagnostic models.

According to one aspect of the invention, the diagnostic model, or diagnostic algorithm, which is fed with more data such as remarkable body temperatures, ambient temperature, a class of personal characteristics, a time of day, can be arranged to additionally use travel data of the examined person to verify whether he or she encountered a sick person or passed through an at-risk region.

According to one aspect of the invention, the system is arranged to operate in the absence of a radar and using RGB cameras to estimate cardiac and respiratory parameters.

According to one aspect of the invention, the temperature relative to a remarkable point may be obtained by time average and by average of the temperatures of a surface defined by pixels from a camera image near the remarkable point. The remarkable point is for example defined geometrically using an image area called Building box, which surrounds it, for example via the geometric mean of the sides of the image area. This image area is a surface delimited by a series of points which is constructed by an algorithm for identifying objects.

According to one aspect of the invention, the system has no RGB camera and/or does not use the temperature-controlled surface. In this case, the system uses the external temperature and a thermal transfer model on the covered areas or only the differences in temperature between the remarkable points.

According to one aspect of the invention, the system is arranged to use a fusion of contactless measurements, in particular of vital signs and of thermal and visible imaging.

The diagnostic information is comprises a class chosen from three predetermined classes, which are “Healthy person”, “Person suspected of disease”, “Person with a high probability of disease”.

The diagnostic information may also comprise an evaluation of the severity of the disease.

The invention also relates to a method for providing diagnostic information for detecting a disease in a person, especially a contagious disease such as COVID-19, this method comprising the following steps:
acquiring examination data on the person, using an acquiring device especially comprising, to acquire these examination data, at least a sensor, such as a radar, for measuring physiology and a thermal camera,
receiving these examination data obtained by the acquiring device,
processing these examination data to obtain diagnostic information of the disease based on an analysis of said examination data,
displaying diagnostic information for the disease based on an analysis of said examination data, this diagnostic information being able to be representative of a probability level of the person having the disease.

The present invention can allow checks in public areas in general, in particular in public thoroughfares, building entrances and exits, at airport gates, in schools.

The present also allows medical monitoring of people, for example for persons who are ill and receiving at-home care.

The invention also relates, independently or in combination with the foregoing, to a system for assisting with provision of diagnostic information, especially with a view to detecting a disease in a person, especially a contagious disease such as COVID-19, this detecting system comprising:
a device for acquiring examination data on the person, this acquiring device especially comprising, to acquire these examination data, at least a sensor, such as a radar, for measuring physiology and a thermal camera, this acquiring device comprising a plurality of sensors arranged to operate without contact with the person, at least one of the contactless sensors being arranged to acquire a response datum provided by the person,
a data processing device arranged to receive these data obtained by several sensors of the acquiring device,
a display device arranged to display diagnostic information for the disease based on an analysis of said data by the data processing device, this diagnostic information especially being able to be representative of a probability level that the person has the disease, this probability level especially being able to be a score.

The invention makes it possible, owing to the plurality of sensors that operate contactless with the person to be examined, to have enough data to establish a more reliable diagnosis.

According to one aspect of the invention, the data processing device comprises an artificial intelligence unit arranged to process the examination data obtained by the acquiring device and to provide said diagnostic information.

According to one aspect of the invention, the data processing device and the display device are part of the same apparatus, for example a computer, in particular a laptop computer.

According to one aspect of the invention, the display device, and optionally also the data processing device, are arranged to be embedded on a motor vehicle.

According to one aspect of the invention, the contactless sensor arranged to acquire a response datum provided by the person comprises a microphone arranged to receive responses provided orally by the person.

According to one aspect of the invention, the contactless sensor may, as a variant of the microphone which operates with voice recognition, comprise means for analyzing the face of the person for example to recognize a YES or a NO, for example by a nod.

Alternatively, the contactless sensor may comprise means enabling the person to point to a response on a screen with a finger, without contact with the screen.

According to one aspect of the invention, the system is arranged to present a questionnaire, for example by displaying questions of the questionnaire on a display screen, or for example using a sound device that reads the questions of the questionnaire aloud, to the person to be examined, and the acquiring device is arranged to receive the responses provided by the person to this questionnaire, for example the microphone being used to record the responses of the person.

According to one aspect of the invention, the system comprises a data storage unit, for example forming part of a computer, arranged to store the questions of the questionnaire.

According to one aspect of the invention, the data processing device is arranged to receive data from the acquiring device relative to the responses provided by the person in response to the questionnaire, and this data processing device is arranged to process these data in combination with data from other sensors of the acquiring device, in order to provide the diagnostic information for the disease.

According to one aspect of the invention, in order to obtain the diagnostic information, the data processing device is arranged to run a diagnostic algorithm based on a fusion of vital data of the person, especially a respiration rate, a respiration depth, an inhalation and exhalation time, a heart rhythm and an arrhythmia, also with questionnaire response data.

According to one aspect of the invention, the system is arranged so as to select the questions to be asked based on the person to be examined, especially on one or more available data on the person.

According to one aspect of the invention, the questions to be asked are medical, useful for improving the diagnosis in combination with the other data.

According to one aspect of the invention, the questions are selected from the following list: Do you have chest pain? Do you have a dry cough? Do you have a sore throat? Do you have a runny nose? Do you have difficulty breathing?

According to one aspect of the invention, the system is arranged to perform a measurement using one of the contactless sensors whereof the result data are compared to a response provided by the person in response to a question of the questionnaire.

This allows the measurement using the sensor to be made more reliable or to be assigned a reliability level.

According to one aspect of the invention, the system is arranged to acquire data relative to a context, for example an ambient temperature.

According to one aspect of the invention, the system is arranged to perform sorting based on a static model to give a score associated with the diagnosis.

According to one aspect of the invention, the system is arranged to carry out the following steps: receiving consent data from the person to undergo the examination, acquiring characterization data of the person such as the gender, age, height, and data representative of the background, acquiring data in response to questions, especially using a microphone, and measurements of vital signs, processing data to allow an evaluation of the reliability level of the diagnostic information, optionally asking additional questions if necessary to refine the diagnostic information, performing sorting based on a static model to give a score associated with the diagnosis.

According to one aspect of the invention, the contactless sensors are arranged to allow measurement acquisition at a distance from the person to be examined, comprised between 50 cm and 2 m.

One object of the invention is also a terminal which includes the assistance system as mentioned above.

This terminal may for example comprise a processing unit arranged to process the data originating from the sensors and to deliver the diagnostic information.

This processing unit may be a computer, with or without a screen on the terminal.

This terminal in particular comprises the various sensors.

This terminal can for example be installed at the entrance to a building.

The invention further relates to a method for assisting with provision of diagnostic information, especially for detecting a disease in a person, especially a contagious disease such as COVID-19, this method comprising the following steps:
acquiring examination data on the person, this acquiring device especially comprising, to acquire these examination data, at least a sensor, such as a radar, for measuring physiology and a thermal camera, this acquiring device comprising a plurality of sensors arranged to operate without contact with the person, at least one of the contactless sensors being arranged to acquire a response datum provided by the person, this sensor especially being a microphone,
receiving these data obtained by several sensors of the acquiring device,
displaying diagnostic information for the disease based on an analysis of said data by the data processing device, this diagnostic information especially being able to be representative of a probability level that the person has the disease, this probability level especially being able to be a score.

The invention and its various applications will be better understood upon reading the following description and examining the accompanying figures:

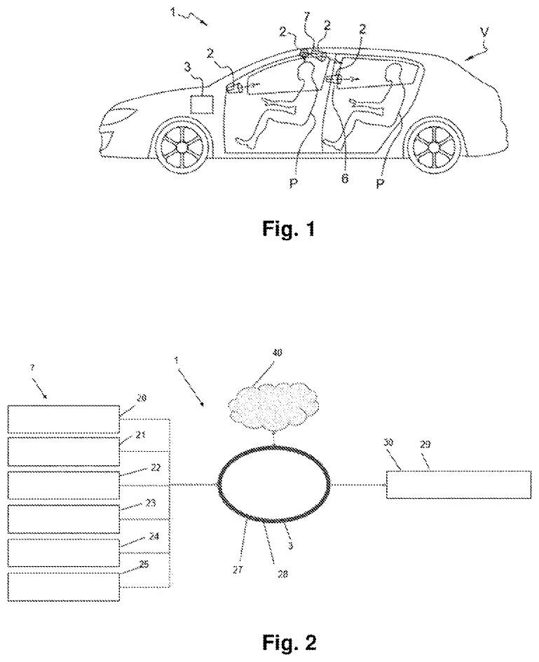

FIG. 1 schematically shows a system according to a non-limiting embodiment of the invention.

FIG. 2 shows a block diagram showing the steps implemented in the system of FIG. 1,

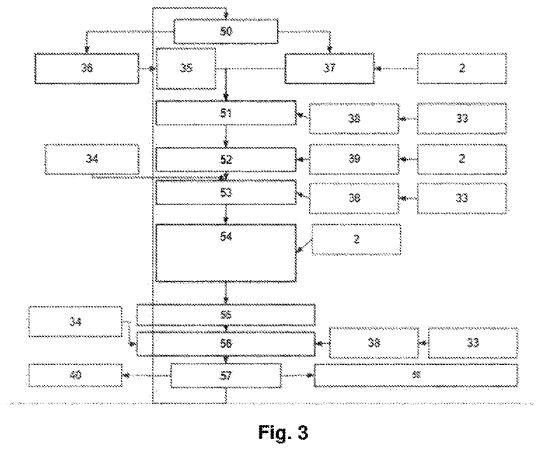

FIG. 3 shows a block diagram showing the steps implemented in a system according to another exemplary embodiment of the invention.

FIGS. 1 and 2 show a system 1 for assisting with the provision of diagnostic information, especially with a view to detecting a disease in a person, especially a contagious disease such as COVID-19, according to the invention.

This system 1 comprises:
an acquiring device 7 for acquiring examination data on the person,
a data processing device 3 arranged to receive these examination data obtained by the acquiring device 7,
a display device 30 arranged to display diagnostic information for the disease based on an analysis of said examination data, this diagnostic information being able to be representative of a probability level that the person has the disease.

This system 1 in particular comprises:
a sensor for the cardiac activity of at least one passenger, here the heart rate, this sensor being a camera operating in the near infrared range,
a sensor for the respiratory activity, in particular in terms of frequency and/or of respiratory depth, of at least one passenger, this sensor being a camera operating in the far infrared range, or a thermal camera,
a radar arranged to measure vital signs of the person,
a sensor for the profile characteristics of the passenger, in particular his or her gender, weight, height and age, this sensor here being a Red Green Blue camera, also called RGB camera,
a card reader for reading an identity card of the person and obtaining personal data of the person.

These sensors and cameras, which form part of the acquiring device 7, are represented by reference 2 in FIG. 1. Some sensors 2 are for example arranged on a ceiling of the vehicle. One of the other cameras 2 is arranged in a lateral upright 6 of the vehicle V.

The heart rate and respiration sensor can be in the seat back or in the center console at the passenger's thigh, this not being limiting.

These sensors 2 are linked for exchanges of information with a data processing device 3 placed on the vehicle V.

The data processing device 3 comprises an artificial intelligence unit arranged to process the examination data obtained by the acquiring device 7 and to provide said diagnostic information.

The data processing device 3 and the display device 30 are part of the same apparatus, for example a computer, in particular a laptop computer.

In this case, the display device 30 is a screen of the laptop computer and the data processing device comprises microprocessor of this computer.

The display device 30 is arranged to be visible by the examined person, and in particular the display device, which in particular comprises a screen, is remote from the data processing device, the display device and the data processing device being for example connected to one another by a wireless link, for example by a 3G, 4G or 5G communication protocol, or by the Internet, or by WiFi, for example.

The display device 30, and also the data processing device 3, here are arranged to be embedded on a motor vehicle.

In a variant, the display device 30, and optionally also the data processing device 3, are arranged to be placed in a fixed manner, in a building or an outer courtyard, for example.

The processing device 3 is arranged to perform the diagnosis automatically, without human intervention.

The device 7 for acquiring examination data is arranged to allow contactless measurements at a safe distance of vital signs as well as thermal and visible imaging on the person to be examined.

The system according to the invention thus advantageously merges contactless measurements at a safe distance of vital signs and thermal and visible imaging.

The artificial intelligence unit is arranged to use a diagnostic model based on artificial intelligence and fed by a reasonable number of clinical measurements.

The invention allows the performance of mobile diagnoses. It is easy to implement, for example using a thermal camera, a physiological measurement sensor such as a radar, and a personal laptop computer.

The acquiring device 7 comprises a radar for acquiring data relating to the vital signs of the person, a thermal camera for temperature measurements providing temperature data, and a camera operating in the visible spectrum for characterizing the tested person providing data characterizing the person.

The data processing device 3 is arranged to run a diagnostic algorithm based on a fusion of vital data, especially a respiration rate, a respiration depth, an inhalation and exhalation time, a heart rhythm and an arrhythmia, an oximetry.

The algorithm uses temperatures measured on remarkable areas, localized by processing the image.

Preferably, these remarkable temperature measurement areas are located inside the mouth, on the tip of the nose, on the cheekbones and on the palm of the hand.

The diagnostic algorithm uses a characterization of the person, such as age, gender, clothing, height, body mass index.

The system is arranged to acquire examination data until the diagnostic information is available within a period of time in particular between 30 and 120 seconds.

The invention thus allows rapid diagnosis without additional delivery time to a physician, for example. The diagnostic information may, if necessary, be sent automatically to a physician and can be stored on a Cloud-type data storage system 40.

The data processing device 3 uses an algorithm for analyzing the acquired examination data and, if applicable, sorting people in order to detect the cases of patients who are sick, based on all of the collected data and artificial intelligence whose first level of learning is carried out on a sample in a hospital environment.

The measurements carried out by the acquiring device can be used to subsequently refine the diagnosis made by the artificial intelligence.

Owing to the invention, a diagnosis can made without contact with the person, which limits the risks of contamination, which is particularly advantageous for example in the case of a pandemic such as that linked to COVID-19.

According to one aspect of the invention, the measured examination data comprise at least one of the following data: temperatures measured at different points of the body of the person to be examined, a respiratory characteristic or a cardiac characteristic.

The acquiring device 7 is arranged to acquire examination data comprising an outside temperature, a temperature measured on a cheekbone of the person, a temperature measured on the tip of the person's nose, and also if necessary, a maximum temperature of the face and a temperature of a clothing item or a reference temperature-controlled surface.

The remarkable measurement points are located by an artificial intelligence via an object identification flowchart.

The identification of the personal characterizations is done by means of Red Green Blue camera (or RGB camera) or Far InfraRed camera (FIR camera) in addition to a reading of the person's identity, owing to a classification system whose training can be carried out on RGB (Red Green Blue) or Infrared images. A larger number of parameters, in particular the age, the gender, the height, the body mass index, the phenotype, for example, are used to improve the diagnostic models.

The diagnostic model, or diagnostic algorithm, which is fed with more data such as remarkable body temperatures, ambient temperature, a class of personal characteristics, a time of day, can be arranged to additionally use travel data of the examined person to verify whether he or she encountered a sick person or passed through an at-risk region.

The temperature relative to a remarkable point may be obtained by time average and by average of the temperatures of a surface defined by pixels from a camera image near the remarkable point. The remarkable point is for example geometrically defined by means of an image area called Building box, which surrounds it. This image area is a surface delimited by a series of points which is constructed by an algorithm for identifying objects.

The diagnostic information is comprises a class chosen from three predetermined classes, which are “Healthy person”, “Person suspected of disease”, “Person with a high probability of disease”.

The invention thus implements the following steps:
acquiring examination data on the person using the acquiring device 7, which are steps 20 to 25 of FIG. 2,
receiving these examination data obtained by the acquiring device,
processing these examination data to obtain diagnostic information of the disease based on an analysis of said examination data, using the data processing device 3 (step 28),
displaying diagnostic information for the disease based on an analysis of said examination data, this diagnostic information being able to be representative of a probability level that the person has the disease (step 29).

Steps 20 to 25 are the following:
the identification of the personal characterizations is done via Red Green Blue cameras (or RGB camera); this is step 20,
acquisition of the person's temperature in step 21 with a thermal camera of the FIR type, the details of this temperature acquisition having already been described,
acquisition of the respiration rate, step 22, using the FIR camera,
acquisition of the heart rate in step 23 using the near-infrared camera, or NIR camera,
acquisition of vital signs using the radar, in step 24,
acquisition of personal data using the card reader in step 25,
potentially oximetry by the near-infrared (NIR) camera.

This also allows medical monitoring of people.

If necessary, the examination data may comprise the size and position of the pupils.

The diagnostic information is sent automatically a Cloud-type remote data storage system.

Similarly, the information useful to the diagnostic model, or diagnostic algorithm, can be received from the Cloud-type remote system.

Potentially two cameras, NIR and FIR, are used. The FIR camera is used for the temperatures and the respiration characteristics and the NIR camera is used for the oximetry and the heart rate.

The acquiring device is especially arranged to acquire examination data comprising an outside temperature, a temperature measured on a cheekbone of the person, a temperature measured on the tip of the person's nose, and also if necessary, a maximum temperature of the face and a temperature of a clothing item or a reference temperature-controlled surface, and if applicable a respiratory volume, tremors, the blood oxygen level.

FIG. 3 shows another exemplary embodiment of the invention.

In this example, the assistance system 1 comprises, like the example described above:
a device 7 for acquiring examination data on the person, this acquiring device especially comprising, to acquire these examination data, at least a sensor, such as a radar, for measuring physiology and a thermal camera, this acquiring device comprising a plurality of sensors 2 arranged to operate without contact with the person, at least one of the contactless sensors being arranged to acquire a response datum provided by the person,
a data processing device 3 arranged to receive these data obtained by several sensors of the acquiring device,
a display device 30 arranged to display diagnostic information for the disease based on an analysis of said data by the data processing device, this diagnostic information especially being able to be representative of a probability level that the person has the disease, this probability level especially being able to be a score.

One of the contactless sensors 2 arranged to acquire a response datum provided by the person comprises a microphone arranged to receive responses provided orally by the person.

The system 1 is arranged to present a questionnaire, for example by displaying questions of the questionnaire on a display screen, or for example using a sound device that reads the questions of the questionnaire aloud, to the person to be examined, and the acquiring device is arranged to receive the responses provided by the person to this questionnaire, here the microphone 33 being used to record the responses of the person.

The system 1 comprises a data storage unit, for example forming part of a computer, arranged to store the questions of the questionnaire 34.

The data processing device 3 is arranged to receive data from the acquiring device relative to the responses provided by the person in response to the questionnaire, and this data processing device is arranged to process these data in combination with data from other sensors of the acquiring device, in order to provide the diagnostic information for the disease.

In order to obtain the diagnostic information, the data processing device 3 is arranged to run a diagnostic algorithm based on a fusion of vital data of the person, especially a respiration rate, a respiration depth, an inhalation and exhalation time, a heart rhythm and an arrhythmia, also with questionnaire response data.

The system is arranged so as to select the questions to be asked based on the person to be examined, especially on one or more available data on the person.

The questions to be asked are medical, useful for improving the diagnosis in combination with the other data.

The system 1 is arranged to perform a measurement using one of the contactless sensors 2 whereof the result data are compared to a response provided by the person in response to a question of the questionnaire.

Different steps of implementing the invention will now be described with reference to FIG. 3.

Step 50 corresponds to the beginning of the method for detecting a disease in a person.

A step 51 follows, wherein the person to be examined inputs data.

In this step 51, the person inputs, for example, data concerning him or her, for example their identity, and then optionally gives their agreement or consent to continue the diagnostic method.

This step 51 can be carried out by the person via a touchscreen 35 coupled to a UV disinfecting device 36. A microphone 33 can be used to record the person's responses to questions. A voice recognition device 38 is clipped to the microphone 33 to enter data taken by the microphone 33 into the data processing unit 3.

Background data 37, for example an ambient temperature, can be acquired by a sensor 2.

In step 52, characteristics related to the person are determined via one or more sensors 2 using an object image recognition system 39, as described in the preceding example.

Additional questions are placed at the end of step 52 to specify the person's clinical status.

For this question/response step 53, the questions are selected for example from the following list: Do you have chest pain? Do you have a dry cough? Do you have a sore throat? Do you have a runny nose? Do you have difficulty breathing?

Responses can be given by the person using the microphone 33.

In the following step 54, the sensors 2 acquire data relative to the vital signs of the person, as described in the preceding example.

Then in step 55, the artificial intelligence provides an initial score representative of the diagnosis obtained based on the data entered thus far.

In step 56, the person is asked additional questions, and the responses obtained are used to refine the initial score obtained in step 55.

In step 57 the artificial intelligence provides a final score, which is recorded in the data storage space 40.

In step 58, the final score is displayed for information to the person.

1. A system for assisting with provision of diagnostic information, for detecting a disease in a person the system comprising:
an acquiring device for acquiring examination data on the person,
wherein the acquiring device comprises a plurality of contactless sensors arranged to operate without contact with the person,
wherein at least one of the contactless sensors is arranged to acquire a response datum provided by the person,
a data processing device arranged to receive the examination and/or response data obtained by a plurality of sensors of the acquiring device,
a display device arranged to display diagnostic information based on an analysis of the examination and/or response data by the data processing device, the diagnostic information representing a probability level of a person having the disease.

2. The system according to claim 1,
wherein the data processing device comprises an artificial intelligence unit arranged to process the examination data obtained by the acquiring device and to provide the diagnostic information.

3. The system according to claim 1,
wherein the data processing device and the display device are part of the same apparatus.

4. The system according to claim 1,
wherein the display device is arranged to be embedded on a motor vehicle.

5. The system according to claim 1,
wherein the contactless sensor arranged to acquire a response datum provided by the person comprises a microphone,
wherein the microphone is arranged to receive responses provided orally by the person.

6. The system according to claim 1,
wherein the system is arranged to present a questionnaire comprising a plurality of questions on a display screen to the person to be examined, and
wherein the acquiring device is arranged to receive responses provided by the person to the questionnaire.

7. The system according to claim 6,
wherein the data processing device is arranged to receive response data from the acquiring device relative to the responses provided by the person in response to the questionnaire, and
wherein the data processing device is arranged to process the response data in combination with data from other sensors of the acquiring device, in order to provide the diagnostic information.

8. The system according to claim 6,
wherein, in order to obtain the diagnostic information, the data processing device is arranged to run a diagnostic algorithm based on a fusion of vital data of the person,
wherein vital data comprises at least one of a respiration rate, a respiration depth, an inhalation and exhalation time, a heart rhythm and an arrhythmia, also with questionnaire response data.

9. The system according to claim 7,
wherein the system is arranged to perform a measurement using one of the contactless sensors,
wherein the result data from the measurement are compared to a response provided by the person in response to a question of the questionnaire.

10. The system according to claim 1,
wherein the system is arranged to carry out the following steps:
receiving consent data from the person to undergo the examination,
acquiring characterization data of the person comprising at least one of gender, age, height, and data representative of the background,
acquiring data in response to questions,
acquiring measurements of vital signs,
processing the acquired data to allow an evaluation of the reliability level of the diagnostic information, and
performing sorting based on a static model to give a score associated with the diagnosis.

11. A method for assisting with provision of diagnostic information for detecting a disease, the method comprising:
acquiring examination data on the person using an acquiring device,
wherein the acquiring device comprises, a sensor,
wherein the acquiring device comprises a plurality of sensors arranged to operate without contact with the person,
wherein at least one of the sensors is arranged to acquire a response datum provided by the person;
receiving the examination and response data obtained by a plurality of sensors of the acquiring device;
displaying diagnostic information for the disease based on an analysis of the examination and response data by the data processing device,
wherein the diagnostic information is representative of a probability level that the person has the disease,
wherein the probability level is a score.

12. The system according to claim 10,
wherein the system is further arranged to ask additional questions to refine the diagnostic information.
